(19) United States
(12) Patent Application Publication
(10) Pub. No.: US 2006/0167474 A1
(43) Pub. Date: Jul. 27, 2006

(54) Apparatus and method for elongation of a papillary muscle

(75) Inventors: Eliot Bloom, Hopkinton, NH (US); Nasser Rafiee, Andover, MA (US); Nareak Douk, Lowell, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/531,832

(22) PCT Filed: Sep. 15, 2004
(86) PCT No.: PCT/US04/30083

§ 371 Date: Jan. 9, 2006

Related U.S. Application Data
(60) Provisional application No. 60/503,051, filed on Sep. 15, 2003.

(51) Int. Cl. A61B 17/122; A61B 17/128; A61F 2/24

(52) U.S. Cl. 606/142; 606/151; 623/2.1

(57) ABSTRACT

A system and method for treating a dilated heart valve by elongating a papillary muscle. The system comprises a delivery catheter 110 and a holding catheter 130. The system further comprises a muscle elongation device 200 including at least two clamping rings 210, 215 slidably connected by at least one connecting rod 220. The muscle elongation device 200 is delivered to a papillary muscle 560 associated with the dilated heart valve, where it is released from the delivery catheter 110 and the clamping rings 210, 215 wrap about and engage the papillary muscle. The muscle tissue is cut between the clamping rings 210, 215, which then move away from each other to a predetermined position, thus permitting the papillary muscle to elongate.

### TECHNICAL FIELD

The technical field of this disclosure is medical devices, particularly, for treating mitral valve regurgitation.

### BACKGROUND OF THE INVENTION

Heart valves, such as the mitral valve, are sometimes damaged by disease or by aging, which can cause problems with the proper function of the valve. Heart valve problems generally take one of two forms: stenosis, in which a valve does not open completely or the opening is too small, resulting in restricted blood flow; or insufficiency, in which blood leaks backward across the valve that should be closed. Valve replacement may be required in severe cases to restore cardiac function.

In various types of cardiac disease, mitral valve insufficiency may result. Any one or more of the mitral valve structures, i.e., the anterior and posterior leaflets, the chordae tendineae, the papillary muscles or the annulus may be compromised by damage from disease or injury, causing the mitral valve insufficiency. Typically, in cases where there is mitral valve insufficiency, there is some degree of annular dilatation resulting in mitral valve regurgitation. Mitral valve regurgitation occurs as the result of the leaflets being moved back from each other by the dilated annulus. Without correction, mitral valve regurgitation may lead to disease progression and/or further annular dilatation and worsening of the insufficiency.

Although mitral valve repair and replacement surgery can successfully treat many patients with mitral valve insufficiency, techniques currently in use are attended by significant morbity and mortality. Most valve repair and replacement procedures require a thoractomy to gain access into the patient's thoracic cavity. Surgical intervention within the heart generally requires isolation of the heart and coronary blood vessels from the remainder of the arterial system and arrest of cardiac function. Open chest techniques with large sternum openings are typically used. Patients undergoing such techniques often have scarring retraction, tears or fusion of valve leaflets as well as disorders of the subvalvular apparatus. It would be desirable, therefore, to provide a method and device for reducing mitral valve regurgitation that would overcome these and other disadvantages.

### SUMMARY OF THE INVENTION

The invention provides an apparatus and method for elongation of a papillary muscle to provide more complete closure of a dilated heart valve. An implantable muscle elongation device can be delivered by a catheter, thus avoiding the significant morbity and mortality associated with open chest surgical techniques used in cardiac valve repair.

A first aspect of the invention provides a system for treating a dilated heart valve comprising a delivery catheter, a holding catheter and a muscle elongation device. The muscle elongation device is held by the holding catheter and received in the delivery catheter, the muscle elongation device including at least two clamping devices slidably connected by at least one connecting rod. When the system is delivered to a papillary muscle associated with the dilated heart valve, the muscle elongation device is released from the holding catheter and the clamping devices wrap about the papillary muscle, the papillary muscle is cut and the clamping devices move away from each other along the at least one connecting rod in response to the tension between the papillary muscle base and the valve annulus.

A second aspect of the invention provides a method for treating a dilated heart valve. The method comprises delivering a muscle elongation device through a lumen of a catheter to a location adjacent a papillary muscle associated with a dilated heart valve. The muscle elongation device having at least two clamping devices disposed along at least one connecting rod is released from the catheter to wrap the clamping devices about the papillary muscle. The method additionally comprises cutting the muscle between the clamping devices and sliding the clamping devices away from each other along the connecting rod.

Yet another aspect of the invention provides a muscle elongation device for treatment of a dilated heart valve. The device comprises at least two clamping devices disposed along at least one connecting rod. The clamping devices clamp a muscle tissue and slide along the connecting rod to create a muscle elongation site.

The foregoing and other features and advantages of the invention will become further apparent from the following detailed description of the presently preferred embodiments, read in conjunction with the accompanying drawings. The drawings are not drawn to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 shows a delivery system for treating a dilated heart valve in accordance with the present invention;

FIG. 2 shows a muscle elongation device for a system for treating a dilated heart valve in accordance with the present invention;

FIG. 3 shows another embodiment of a delivery catheter for a system for treating a dilated heart valve in accordance with the present invention;

FIGS. 4 to 7 illustrate the placement of the device of FIGS. 1 to 2; and

FIG. 8 is a flowchart illustrating a method of elongation of a papillary muscle in accordance with another aspect of the invention.

### DETAILED DESCRIPTION OF THE PRESENTLY PREFERRED EMBODIMENT

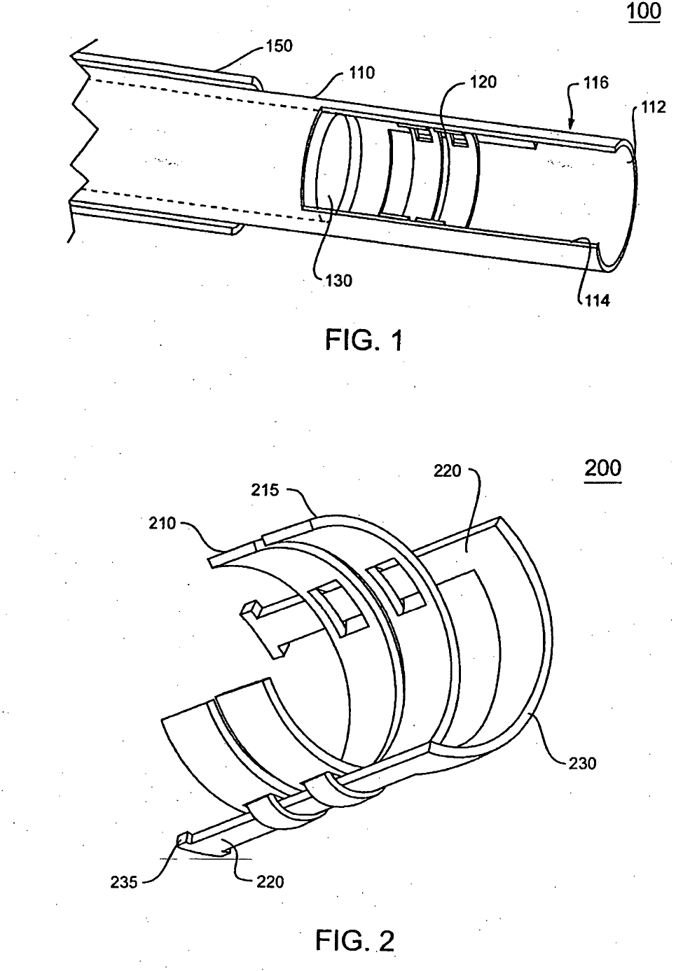

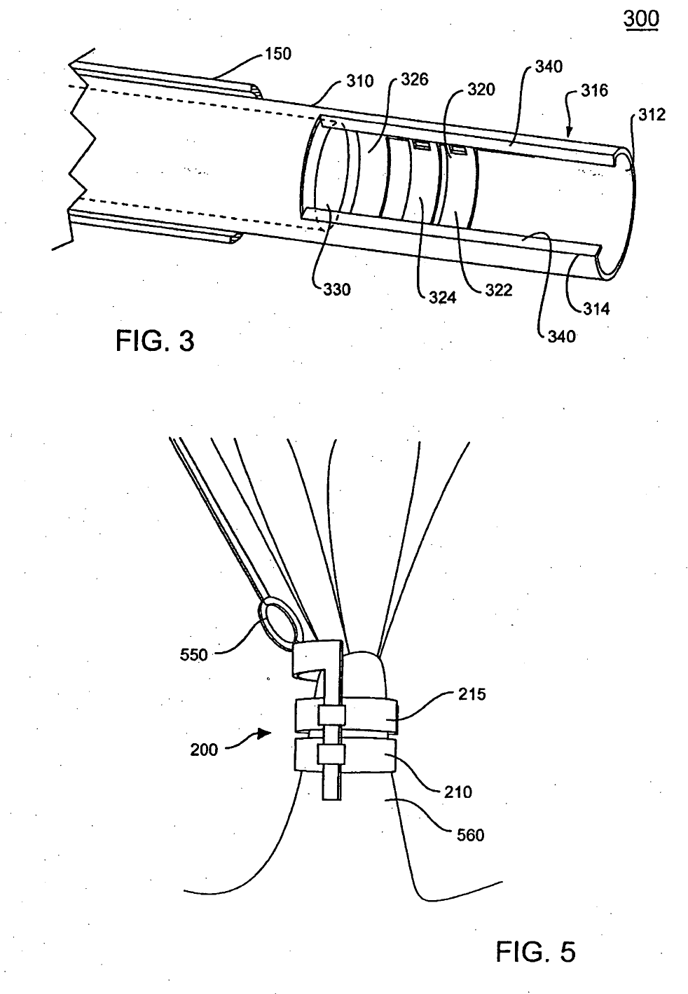

FIGS. 1-2 illustrate a system for treating a dilated heart valve by deploying a muscle elongation device to a papillary muscle. The muscle elongation device can be delivered percutaneously through a delivery catheter using a holding catheter or other mechanical means to deploy and expand the muscle elongation device. Alternatively, the muscle elongation device can be delivered surgically using any known surgical technique including, but not limited to, thoracotomy, sternotomy and open cardiac surgical techniques.

FIG. 1 illustrates delivery catheter 110 used to deploy the system disclosed herein at 100. The invention may be practiced, however, with any appropriate means for delivering the device to a desired location for papillary muscle elongation. In one example, the device is implanted in the left ventricle via the aorta (see FIG. 6). In one embodiment, a guide catheter 150 provides a pathway for advancing delivery catheter 110 to the target muscle. The use of guide catheters are well known to those with skill in the art.

Those skilled in the art will appreciate that numerous paths are available to gain access to a papillary muscle site. For surgical approaches with an open chest or open heart, a trocar or cannula may be inserted directly in the superior vena cava or the aortic arch. The delivery element can then follow the same path as the percutaneous procedure to reach the left ventricle, either transeptally or through the cardiac valves. Transeptal approaches, whether percutaneous or surgical, may require placement of a closure device at the transeptal puncture on removal of the delivery element after the procedure. Similar percutaneous or surgical approaches can be used to access the other cardiac valves, if the muscle elongation device is to be implanted on a papillary muscle for a cardiac valve other than the mitral valve.

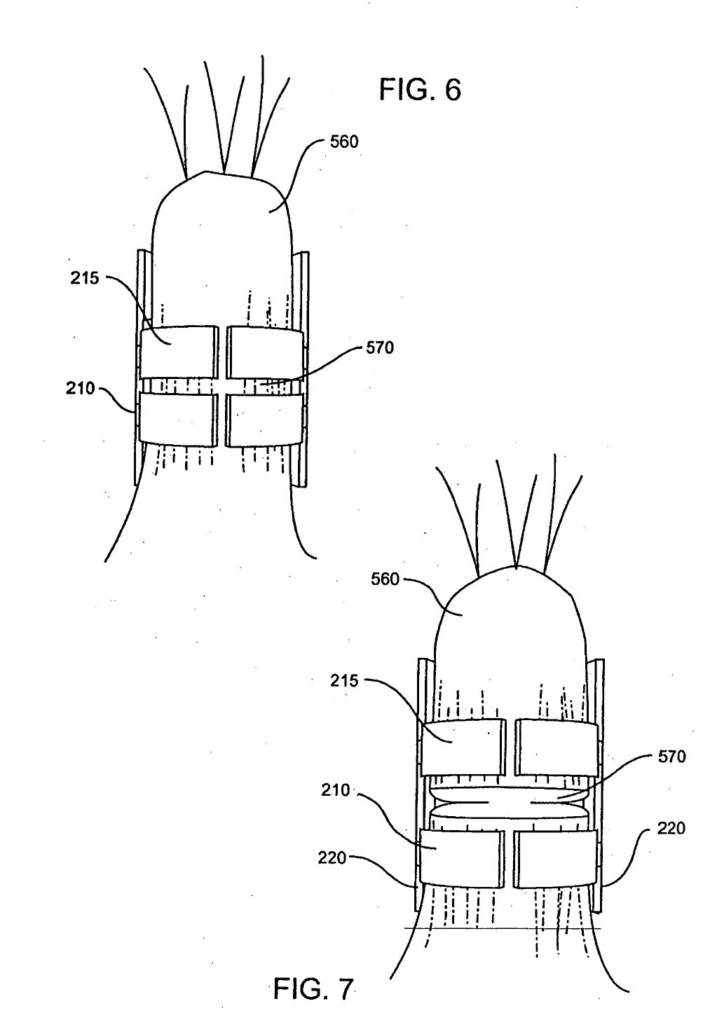

Delivery catheter 110 having lumen 112 is first inserted to provide a path for the muscle elongation device 120 from the exterior of the patient to the left ventricle (see FIG. 4). Holding catheter 130 releasably holds muscle elongation device 120 during advancement through delivery catheter lumen 112 to position muscle elongation device 120 for deployment at the desired location. Holding catheter 130 may also serve as a conduit for electrical current and may grip or release in response to an applied current. In one embodiment, holding catheter 130 is a push rod for deploying muscle elongation device 120 from delivery catheter 110.

In another embodiment illustrated in FIG. 5, holding catheter 130 comprises a gripping device 550. The gripping device may comprise forceps used to deliver the elongation device pictured in FIG. 2, and may be delivered through lumen 112 of delivery catheter 110. In one embodiment, forceps are modified biopsy forceps that releaseably and securely grip muscle elongation device 120. In other embodiments, forceps may also serve as a conduit for electrical current and may grip or release in response to an applied current. Forceps may also include a controller (not shown) used to control the grip or release of the forceps.

Delivery catheter 110 includes side delivery port 114 at distal end 116. Side delivery port 114 provides an opening for placing at least a portion of the target muscle within the distal end 116 of delivery catheter 110 as shown in FIG. 4.

A locating device may be used to assist in accurate placement of the system disclosed herein. In one embodiment, the locating device may comprise a guide wire, as is known to those of ordinary skill in the art. In other embodiments, the locating device may comprise a soft balloon for positioning the distal end 116 of delivery catheter 110 in the apex of the ventricle. In yet other embodiments, the locating device may be a radio-opaque coating on delivery catheter 110 to assist in fluoroscopic imaging of the catheter. Although these locating devices are not shown in the attached figures, these devices are known to those of skill in the art, and further discussion is not warranted.

FIG. 2 shows muscle elongation device 200 in accordance with one embodiment of the invention. Device 200, as shown, comprises two clamp rings 210, 215 and two connecting rods 220. Alternatively, muscle elongation device 200 may comprise more than two clamp rings and one or more connecting rods 220. As shown, a first clamp ring 210 is fixed between the two connecting rods 220, and a second clamp ring 215 is slidably mounted along the connecting rods 220. Connecting rods 220 are provided with stop 230 to prevent the second clamp ring 215 from sliding off the ends of connecting rods 220. In one embodiment, stop 230 comprises enlarged ends of connecting rods 220. In another embodiment, connecting rods 220 may include stops 235. Stops 235 may be utilized with embodiments of muscle elongation device 200 having a first clamp ring 210 that is slidably mounted on connecting rods 220. In yet another alternative, muscle elongation device 200 may comprise one slidable clamping ring 215, stops 235 positioned at each end of the connecting rods 220 and stop 230, where stop 230 acts as a fixed clamping ring. In one embodiment, ratchet teeth (not shown) are disposed along connecting rods 220 to prevent second clamp ring 215 from sliding along connecting rods 220 towards first clamp ring 210 after deployment. FIG. 2 illustrates device 200 in a pre-deployment or delivery configuration for passage through delivery catheter 110. In this configuration, muscle elongation device 200 has a C-shaped cross section with a slight axial separation between the two clamp rings 210, 215.

Clamp rings 210, 215 are composed of a biocompatible material comprising a metallic or a polymeric base. The material may be, for example, stainless steel, nitinol, tantalum, cobalt nickel alloy, platinum, titanium, a thermoplastic or thermoset polymer, or a combination thereof. In some embodiments, clamp rings 210, 215 comprise an elastic shape-memory material, such that clamp rings 210, 215 may be formed to assume a certain shape upon release of a constraining force. In such an embodiment, discussed below and shown in FIG. 5, clamp rings 210, 215 are formed to assume a clamping configuration. The clamping configuration has a substantially closed circular or ring shaped cross section that is assumed after being restrained in an open shape (the delivery configuration). In other embodiments, clamp rings 210, 215 may comprise a thermal shape-memory material that will assume the desired end shape, clamping configuration, only with the application of heat, as by resistance heating with electrical current. In either embodiment, clamp rings 210, 215 assume the clamping configuration of a ring or circular shape after delivery of the clamping device to the desired region of the papillary muscle. Clamp rings 210, 215 have a first diameter when in the delivery configuration and a second diameter in the clamping configuration. The second diameter is less than the first diameter to effectively wrap around the target muscle. In one embodiment, clamp rings 210, 215 are between 6 and 9 millimeters in diameter when in the clamping configuration. Clamp rings 210, 215, as shown, are rectangular in cross-section. In one embodiment, the material comprising clamp rings 210, 215 has a thickness of 0.005 to 0.010 inches (0.127 to 0.254 mm). In other embodiments, the cross-section of clamp rings 210, 215 may be square, triangular or any other appropriate shape.

Connecting rods 220 comprise a biocompatible material having a metallic or polymeric base. The material may be, for example, stainless steel, nitinol, tantalum, cobalt nickel alloy, platinum, titanium, a thermoplastic or thermoset polymer, or a combination thereof. In one embodiment, connecting rods 220 are rectangular in cross section having a thickness of 0.005 to 0.010 inches (0.127 to 0.254 mm). In one embodiment, the diameter of connecting rods 220 is less than the thickness of clamping devices 210, 215. In another embodiment, connecting rods 220 are rectangular or square in cross-section.

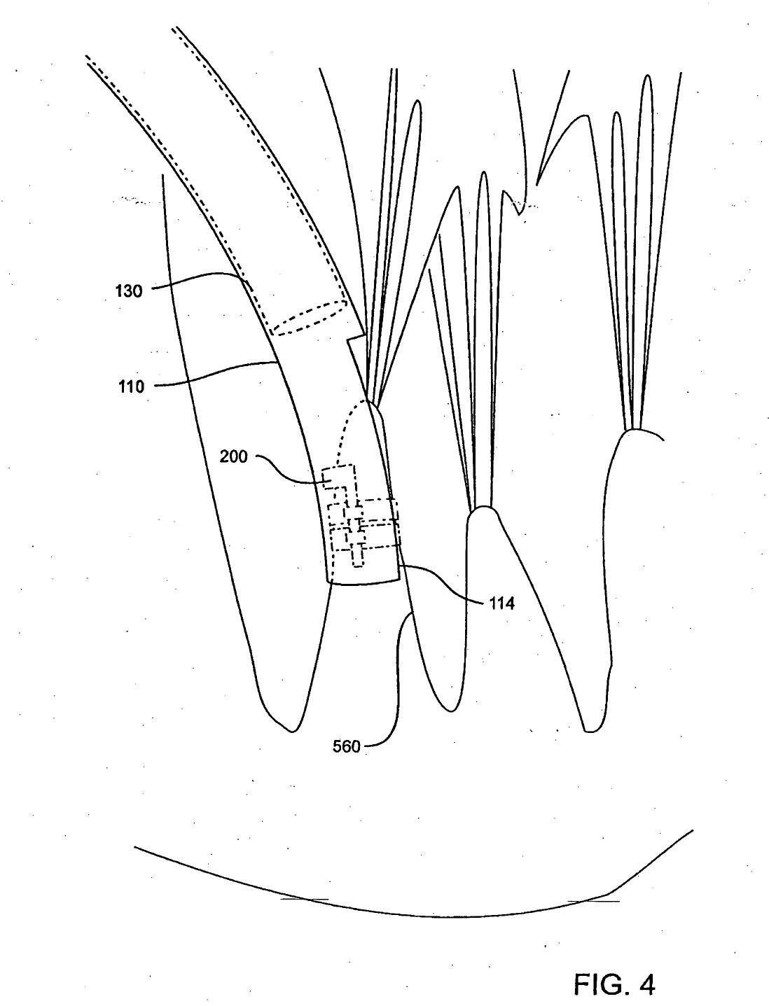

FIG. 3 illustrates another embodiment of a delivery system 300 for delivering a muscle elongation device, in accordance with the present invention. Delivery system includes delivery catheter 310, muscle elongation device 320 and holding catheter 330. Muscle elongation device 320 includes clamp rings 322, 324, connecting rods (not shown) and stop 326. In this embodiment, muscle elongation device 320 is composed of an elastic shape-memory material, such that clamp rings 322, 324 may be formed to assume a certain shape upon release of a constraining force. Clamp rings 322, 324 may be formed to assume a substantially closed circular or ring shape after being restrained in an open shape. Delivery catheter 310 includes restraining members 340 for providing a constraining force to muscle elongation device 320. Restraining members 340 comprise elongate members extending substantially perpendicularly from the edge of side delivery port 314. Restraining member 340 provides the constraining force for maintaining the delivery configuration until muscle elongation device 320 is deployed.

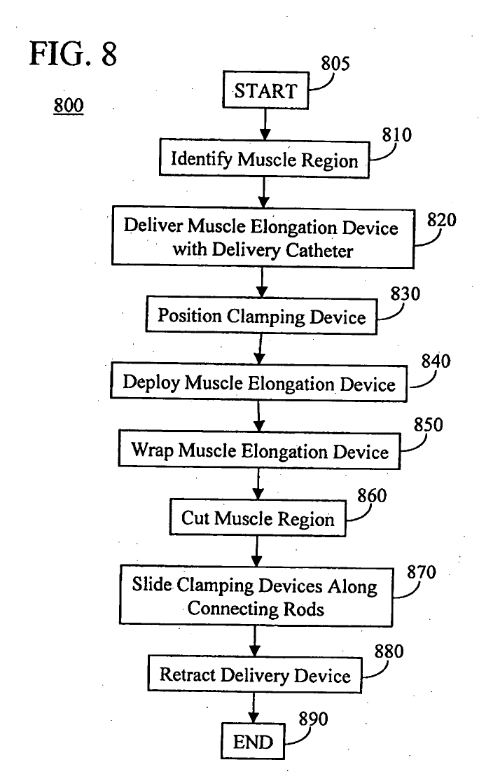

FIGS. 4-8 illustrate a method of using a muscle elongation device, in accordance with the present invention. FIGS. 4-7 illustrate the delivery and placement of the muscle elongation device. FIG. 8 is a flow chart illustrating a method of using the device shown in FIGS. 1-3 in accordance with another aspect of the invention at 800. Method 800 begins at step 805.

First, a papillary muscle is identified as being associated with a dilated heart valve (Block 810).

Second, the muscle elongation device of FIGS. 1-2 is delivered to a region of the targeted papillary muscle (Block 820). Any appropriate technique for accessing the interior of a ventricle and papillary muscles may be used. A variety of appropriate techniques is known to those of ordinary skill in the art and no further discussion is warranted. The muscle elongation device disclosed herein may be delivered through delivery catheter 110, and a practitioner may find the aorta or vena cava to be advantageous approaches, though not an element of the invention. Other approaches are briefly discussed above in the discussion of FIG. 1. In one embodiment, a guide catheter is placed for advancement of the delivery catheter to the target muscle.

Referring to FIG. 4, side delivery port 114 permits delivery catheter 110 to be positioned around the targeted muscle region, thereby placing clamp rings 210, 215 also in a position around the targeted muscle region (Block 830). At delivery, the clamping devices are in the open delivery configuration, so the muscle elongation device is as pictured in FIG. 2.

Next, muscle elongation device 200 is deployed from delivery catheter 110 (Block 840). In one embodiment, the device is deployed by pushing the device from delivery catheter 110 using axial force applied to holding catheter 130. Alternatively, elongation device 200 may be held in place by holding catheter 130 while delivery catheter 110 is withdrawn. In another embodiment, holding catheter 130 may be a forceps 550, as seen in FIG. 5, instead of holding catheter 130 illustrated in FIG. 1. In another embodiment, device 200 is deployed by retracting delivery catheter 110 from surrounding muscle elongation device 200.

Referring to FIG. 5, once deployed, muscle elongation device 200 clamps around the papillary muscle 560 (Block 850). In one embodiment of the invention, the muscle elongation device 200 comprises a shape memory material such as nitinol and upon deployment from delivery catheter 110 (Block 840), the clamp rings 210, 215 wrap and clamp around the muscle in the clamping configuration, as shown in FIG. 6. Use of elastic shape-memory materials allows the clamp rings 210, 215 to wrap around the muscle by assuming the shape that has been preformed into the material. In other embodiments of the invention, an electric current is applied to the device to cause the clamp rings 210, 215 to wrap and clamp around the muscle. In those embodiments, forceps 550 may provide the conduit for conducting the necessary electrical current.

Referring to FIG. 6, the papillary muscle 560 is cut or severed at 570 between clamp rings 210, 215 (Block 860). In one embodiment, the muscle is cut with a surgical blade. In another embodiment, the muscle is cut by an electrical current applied by the forceps. In another embodiment, the muscle is cut by any appropriate cutting tool, such as a laser.

Next, clamp ring 215 slides along the connecting rods 220 and away from clamp ring 210 (Block 870). Tension applied by normal cardiac movement will slide rings 210, 215 apart and provide elongation of the papillary muscle. At this step, the device appears generally as illustrated in FIG. 7. Sliding clamp rings 210, 215 apart provides separation of the cut muscle sections to elongate the papillary muscle. Alternatively, the clamp rings may be slid along the connecting rods by forceps 550.

Finally, the catheter and gripping device are retracted from the body, leaving the device surrounding the muscle in the clamping configuration (Block 880). The elongated muscle tissue is allowed to form scar tissue around the device. Method 800 ends at Block 890.

FIG. 7 depicts the muscle elongation device deployed upon the posterior papillary muscle 560. The illustration of treatment of the posterior papillary muscle in no way limits the invention, as the device may be employed on any papillary muscle, and indeed, the device may be used on any appropriate muscle tissue. As shown in FIG. 7, clamp rings 210, 215 wrap around the posterior papillary muscle and are connected by connecting rods 220. In FIG. 7, two connecting rods are shown, although any number of connecting rods may be used to practice the invention.

It is important to note that FIGS. 1-8 illustrate specific applications and embodiments of the present invention, and are not intended to limit the scope of the present disclosure or claims to that which is presented therein. For example, the muscle elongation system of the present invention can be used for other heart valves, such as a tricuspid valve, in addition to the mitral valve. The muscle elongation system of the present invention may also be used on muscles other than a papillary muscle. Different arterial and venous approaches can also be used. Upon reading the specification and reviewing the drawings hereof, it will become obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for treating a dilated heart valve comprising:
a delivery device 100 comprising a delivery catheter 110 and a holding catheter 130;
a muscle elongation device 200 coupled to the holding catheter 130 and received in the delivery catheter 110, the muscle elongation device 200 including at least one clamping device 215 and disposed adjacent a distal end 116 of the holding catheter 110, the at least one clamping device 215 slidably disposed on an at least one connecting rod 220, wherein when the system is delivered to a muscle region associated with the dilated heart valve, the muscle elongation device 200 is released from the delivery catheter 110 and the at least one clamping device 215 wraps around the muscle region.

2. The system of claim 1 wherein the muscle elongation device 200 includes a first clamping device 210 fixedly attached to the at least one connecting rod 220 and a second clamping device 215 slidably disposed on the at least one connecting rod 220.

3. The system of claim 1 wherein the delivery catheter further comprises a side delivery port 114 located adjacent the distal end 116 of the delivery catheter 110.

4. The system of claim 3 wherein the side delivery port 114 further comprises two restraining members 340.

5. The system of claim 1 further comprising a locating device.

6. The system of claim 5 wherein the locating device comprises a balloon.

7. The system of claim 5 wherein the locating device comprises a guide wire.

8. The system of claim 1 wherein the holding catheter comprises biopsy forceps 550.

9. The system of claim 1 wherein the at least one clamping device 210, 215 comprise a shape-memory material.

10. The system of claim 9 wherein the shape-memory material is an elastic shape-memory material.

11. The system of claim 9 wherein the shape-memory material is a thermal shape-memory material.

12. The system of claim 9 wherein the shape-memory material is a material chosen from a group consisting of stainless steel, nitinol, tantalum, cobalt nickel alloy, platinum, titanium, a thermoplastic or thermoset polymer, or a combination thereof.

13. The system of claim 1 wherein the connecting rod 220 comprises an at least one stop 230 disposed at a proximal end of the connecting rod.

14. The system of claim 13 wherein the connecting rod 220 comprises a second stop 235 disposed at a distal end of the connecting rod.

15. A muscle elongation device 200 for treatment of a dilated heart valve, comprising:
at least one connecting rod 220;
a first clamping device 210 fixed to the at least one connecting rod; and
a second clamping device 215 slidably disposed along the connecting rod,
wherein the first clamping device 210 and the second clamping device 215 have a first diameter in a delivery configuration and a second diameter in a clamping configuration, the second diameter less than the first diameter.

16. The muscle elongation device of claim 15 further comprising:
at least one stop 230 disposed on the at least one connecting rod 220.

17. The muscle elongation device of claim 15 wherein the muscle elongation device 200 is composed of a shape memory material.

18. The muscle elongation device of claim 17 wherein the shape memory material is an elastic shape memory material.

19. The muscle elongation device of claim 17 wherein the shape memory material is a thermal shape memory material.

20. The muscle elongation device of claim 17 wherein the shape-memory material is a material chosen from a group consisting of stainless steel, nitinol, tantalum, cobalt nickel alloy, platinum, titanium, a thermoplastic or thermoset polymer, or a combination thereof.

21. A method for treating a dilated heart valve, the method comprising:
delivering a muscle elongation device 200 in a lumen of a delivery catheter 110 proximate a dilated heart valve;
positioning at least two clamping devices 210, 215 disposed along at least one connecting rod 220 of the muscle elongation device 200 on a muscle region 560 proximate the dilated heart valve;
releasing the muscle elongation device 200 from the delivery catheter 110;
wrapping the clamping devices 210, 215 about the muscle region 560;
cutting the muscle between the clamping devices 210, 215; and
sliding the clamping devices 210, 215 away from each other along the connecting rod.

22. The method of claim 21 further comprising locating the cardiac muscle with a location device.
